(19) United States
(12) Patent Application Publication
(10) Pub. No.: US 2025/0121179 A1
(43) Pub. Date: Apr. 17, 2025

(54) HEART PUMP ASSEMBLY WITH A PUMP HOUSING CONFIGURED TO REDUCE HEMOLYSIS

(71) Applicant: Abiomed, Inc., Danvers, MA (US)

(72) Inventors: Zhongwei Qi, Danvers, MA (US); Scott C. Corbett, Danvers, MA (US)

(73) Assignee: Abiomed, Inc., Danvers, MA (US)

(21) Appl. No.: 18/912,736

(22) Filed: Oct. 11, 2024

Related U.S. Application Data
(60) Provisional application No. 63/589,719, filed on Oct. 12, 2023.

(51) Int. Cl. A61M 60/90; A61M 60/13; A61M 60/81

(52) U.S. Cl. CPC A61M 60/90; A61M 60/13; A61M 60/81; A61M 2205/04; A61M 2205/10; A61M 2206/20

(57) ABSTRACT

A percutaneously insertable blood pump assembly comprises a pump section, a catheter attached to a proximal end of the pump section, a first apertured section distal to the pump section and in fluid communication therewith, a cannula affixed to and in fluid communication with the first apertured section, and a second apertured section distal to the cannula and in fluid communication therewith. One of the first and second apertured section is a blood outlet from the cannula. The blood outlet has a body portion, an apertured portion, and a ring portion. The plurality of struts in the apertured portion extend from and join the body portion to the ring portion. The struts are asymmetric along at least a portion of their length from the body portion of the outlet to the ring portion of the outlet.

### CROSS REFERENCE TO RELATED APPLICATIONS

The present application claims priority to U.S. Provisional Application No. 63/589,719 filed Oct. 12, 2023, the disclosure of which is hereby incorporated by reference herein in its entirety.

### TECHNICAL FIELD

A heart pump assembly is described herein, and more particularly, a heart pump assembly having a pump housing configured to reduce hemolysis while in use.

### BACKGROUND

A heart pump, such as a percutaneous intracardiac heart pump assembly, can be inserted into a heart to deliver blood from the heart into an artery. When deployed in the heart, a heart pump assembly pulls blood from the left ventricle of the heart and expels blood into the aorta, or pulls blood from the right ventricle and expels blood into the pulmonary artery. Specifically, for left ventricle support, the blood enters the heart pump assembly via a blood inlet located at a distal end of the heart pump assembly, travels through a cannula of the heart pump assembly, and expels via a plurality of outlets defined on the pump housing located at a proximal end of the heart pump assembly.

### BRIEF SUMMARY

Described herein is a percutaneously insertable blood pump assembly having a pump section, a catheter attached to a proximal end of the pump section, a first apertured section distal to the pump section and in fluid communication therewith, a cannula affixed to and in fluid communication with the first apertured section, and a second apertured section distal to the cannula and in fluid communication therewith. One of the first and second apertured section may be a blood outlet from the cannula. The blood outlet may have a body portion, an apertured portion, and a ring portion. The plurality of struts in the apertured portion may extend from and join the body portion to the ring portion. The struts may be asymmetric along at least a portion of their length from the body portion of the outlet to the ring portion of the outlet.

In one alternative aspect, the blood outlet may be the first apertured section may be the blood outlet. In another alternative aspect, the blood outlet may be the second apertured section.

In any of the above aspect, the asymmetric struts have a cross-section having a leading edge and a trailing edge, the leading edge forming a first angle and the trailing edge forming a second angle. In any of the above aspects, the body portion of the blood outlet may have an outer perimeter and an inner perimeter and the leading edge of the strut may be removed from the outer perimeter of the body portion and the trailing edge portion may be removed from the inner perimeter of the body portion. In any of the above aspects, the first angle and the second angle form acute angles. In one aspect, the first angle may be about 10 degrees to about 70 degrees. In a further aspect, the first angle may be about 20 degrees to about 45 degrees. In a further aspect, the first angle may be about 30 degrees to about 40 degrees.

In another aspect, the second angle may be about 5 degrees to about 45 degrees. In a further aspect, the second angle may be about 20 degrees. In a further aspect, at least one of the leading edge and the trailing edge are filleted or rounded or a combination of filleted and rounded. In one aspect the, fillet has a radius of about 1.25 mm.

In any of the above aspects, both the leading edge and the trailing edge are filleted or rounded or a combination of filleted and rounded. In a further aspect, the struts have a hydrofoil-like cross-section along at least a portion of their length from the body portion of the outlet to the ring portion of the outlet. In an alternative of any of the above aspects, the struts have a hydrofoil-like cross-section along an entire length from the body portion of the outlet to the ring portion of the outlet.

In any of the above aspects, the plurality of apertures may have dimensions and the dimensions of each aperture are about the same.

In any of the above aspects, percutaneously insertable blood pump assembly may have an atraumatic tip distal to the second apertured section.

### BRIEF DESCRIPTION OF DRAWINGS

FIG. 1 is a perspective view of a heart pump assembly, according to an aspect of the assembly described herein.

FIG. 2 is a partial perspective view of an apertured end portion of the pump housing of the heart pump assembly of FIG. 1.

FIG. 3 is a partial cross-sectional view of the apertured end portion of the pump housing of FIG. 2 along line A-A at the intersection of a strut and the body of the end portion of the pump housing.

FIG. 4 is a partial perspective view of an exemplary prior art apertured end portion of a pump housing of a heart pump assembly.

FIG. 5 shows blood flow recirculation at the outlets of the apertured portion of the pump housing of FIG. 4.

FIG. 6 shows blood flow recirculation at the outlets of the apertured portion of the pump housing of FIG. 2.

FIG. 7 shows wall shear stress as blood passes through the outlets of the apertured portion of the pump housing of FIG. 4.

FIG. 8 shows wall shear stress as blood passes through the outlets of the apertured portion of the pump housing of FIG. 2.

### DETAILED DESCRIPTION

Aspects of the present disclosure are described in detail with reference to the drawing figures wherein like reference numerals identify similar or identical elements. It is to be understood that the disclosed aspects are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The heart pump assembly may be percutaneously inserted into the heart through the aorta. The blood inlet may be positioned past the aortic valve in the left ventricle, in order to pull blood from the left ventricle and expel the blood into the aorta. The blood enters the heart pump assembly via a blood inlet located at a distal end of the heart pump assembly, travels through a cannula of the heart pump assembly, and expels via a plurality of outlets defined on the pump housing located at a proximal end of the heart pump assembly. The inventors have recognized and appreciated that the design of the currently available pump housing of a heart pump assembly produces high wall shear stress on blood and large blood flow recirculation as the blood expels through the outlets of the pump housing, thereby causing hemolysis in the bloodstream.

The heart pump assembly described herein provides a heart pump assembly with an improved pump housing. The pump housing may be configured and designed to prevent or reduce wall shear stress of blood and prevent large blood flow recirculation as the blood passes through the outlets of the pump housing. The reduction in wall shear stress and large blood flow recirculation thereby reduces hemolysis in the bloodstream, as will be described in detail below.

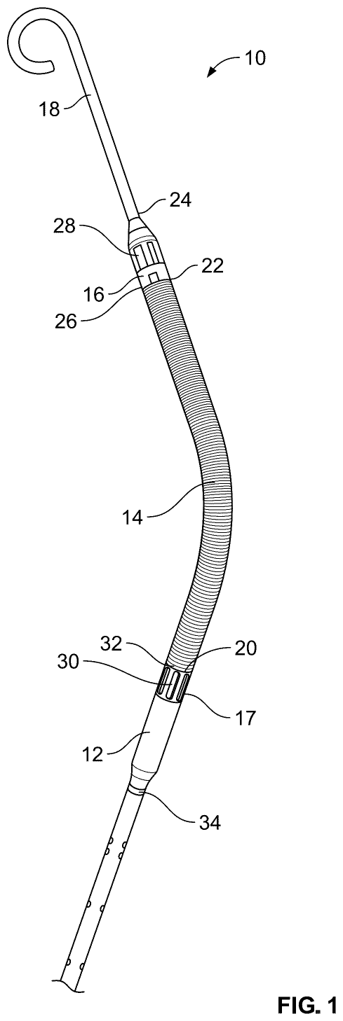

FIG. 1 illustrates a heart pump assembly 10 comprising an improved apertured portion of pump housing 12 in accordance with the present technology. With reference to the embodiment illustrated in FIG. 1, the apertured portion of the pump housing may be the blood outlet 17. The blood outlet 17 is illustrated at the distal end of pump housing 12. The blood outlet 17 may be monolithic with or attached to the pump housing 12. The heart pump assembly 10 includes the pump housing 12 with apertures or outlet openings 30 in the blood outlet 17, a cannula 14, a blood inlet 16, and an atraumatic tip 18 for stabilizing the heart pump assembly 10 in the ventricle of the heart. The heart pump assembly 10 can vary in any number of ways. For example, the embodiment of FIG. 1 herein may include a motor (not shown) within the pump housing 12 or may be positioned outside of a patient's body and operatively coupled to a rotor (not shown) via a drive shaft or cable (now shown).

Referring again to FIG. 1, the cannula 14 extends between a proximal end 20 and a distal end 22 thereof. At the distal end 22 of the cannula 14, the cannula 14 and blood inlet 16 are connected and are in fluid communication with each other. The proximal end 20 of the cannula 14 may also be connected to the blood outlet 17 of the pump housing 12. The blood inlet 16 is may also be connected to the atraumatic tip 18. Thus, the blood inlet 16, cannula 14, pump housing 12, and atraumatic tip 18 are all connected to form the heart pump assembly 10. The pump housing 12, blood inlet 16, cannula 14, and blood outlet 17 are all in fluid communication with each other. In other aspects, e.g., for blood pumps positioned in the right side of the heart, the blood inlet may be proximal and the blood outlet may be distal, relative to the insertion site for the blood pump.

In the illustrated aspect, the blood inlet 16 extends between a distal end 24 and a proximal end 26 thereof. The blood inlet 16 includes a plurality of openings 28 defined and positioned between the distal and proximal ends 24, 26 for allowing blood to enter therethrough and to travel through the cannula 14. The atraumatic tip 18 may be connected at the distal end 24 of the blood inlet 16, as shown in FIG. 1.

The atraumatic tip 18 may be shaped as a flexible extension having a curved end portion as shown in FIG. 1. Alternatively, the atraumatic tip 18 may be configured as a straight extension or as a ball. Also, the atraumatic tip 18 may include a lumen for the passage of a guidewire through the atraumatic tip 18. The atraumatic tip 18 acts as a mechanical spacer that provides spacing between the plurality of openings 28 of the blood inlet 16 and the tissue that forms the inner surface of the heart. This spacing prevents the plurality of openings 28 from suctioning to the walls of the heart, heart valves (e.g., the mitral valve), or any other anatomical structure in the heart. This may reduce the risk of blockage of one or more of the plurality of openings 28 of the blood inlet 16 and may reduce or prevent damage to the heart tissue.

The pump housing 12 is configured to accommodate an impeller (not shown) and a motor (not shown) therewithin. The motor is used to rotate the impeller to draw blood from the heart into the heart pump assembly 10. Specifically, rotation of the blades of the impeller creates suction through the cannula 14 for the blood to flow into the heart pump assembly 10. The blood enters the cannula 14 and travels therethrough and exits the heart pump assembly 10 from a plurality of outlet openings 30 defined in the outlet portion 17 of the pump housing 12 positioned at the proximal end of the cannula 14.

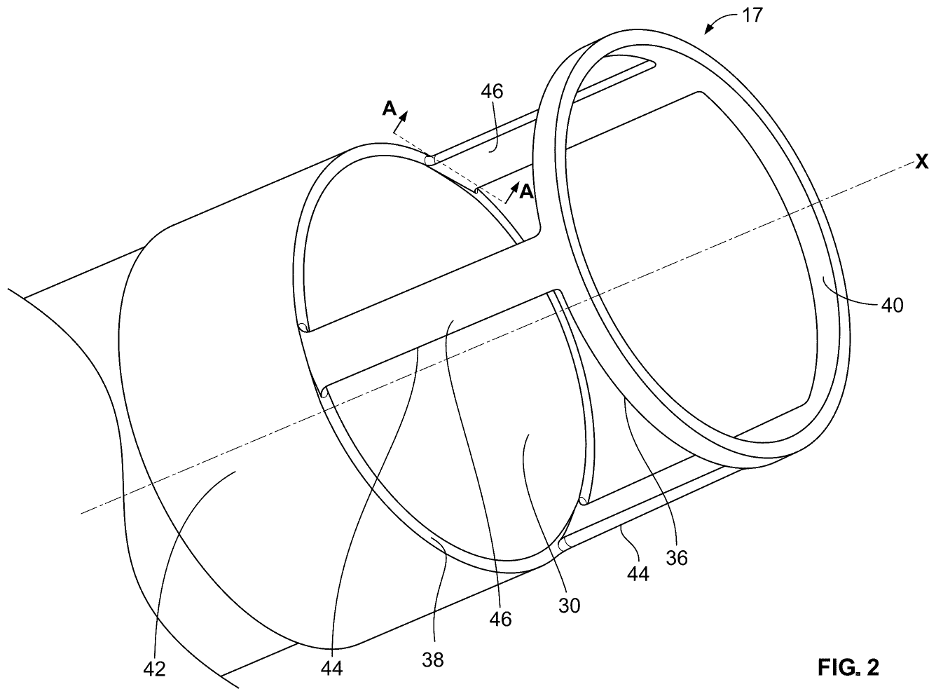

Referring to FIGS. 1 and 2, the pump housing 12 extends between a distal end 32 and a proximal end 34 thereof, and includes the outlet portion 17 plurality of outlet openings 30 defined and positioned between the distal and proximal ends 32, 34. The plurality of outlet openings 30 are oriented radially around a circumference of the outlet portion 17 of the pump housing 12 with a substantially evenly spaced distance between each of the plurality of outlet openings 30. Each of the plurality of outlet openings 30 has an associated length measured parallel to an axis X, a width measured radially with respect to the X axis, and an area. For example, each outlet opening 30 has a length, a width, and an area through which blood from the cannula 14 may exit the heart pump assembly 10. In one aspect, each of the plurality of outlet openings 30 are similarly dimensioned (e.g., length, width, and area) such that the shape and size of the outlet openings 30 are substantially identical. The plurality of outlet openings 30 may have any suitable shape to allow blood to exit the heart pump assembly 10. For example, the plurality of outlet openings 30 may be oblong, oval, square, tear-shaped, round or any other suitable shape.

Referring particularly to FIG. 2, each of the plurality of outlet openings 30 may be bounded by edges 36 and 38 and are separated from each other by struts 46. Collectively, the edges 36 define an end ring 40 of the outlet portion 17 of the pump housing 12. Collectively, edges 38 define an end 42 of the outlet portion 17 of the pump housing 12 from which the outlet openings 30 and the struts 46 extend. The struts 46 have outer edges 44. In one aspect, the outer edges 44 are either filleted or rounded or a combination of filleted and rounded.

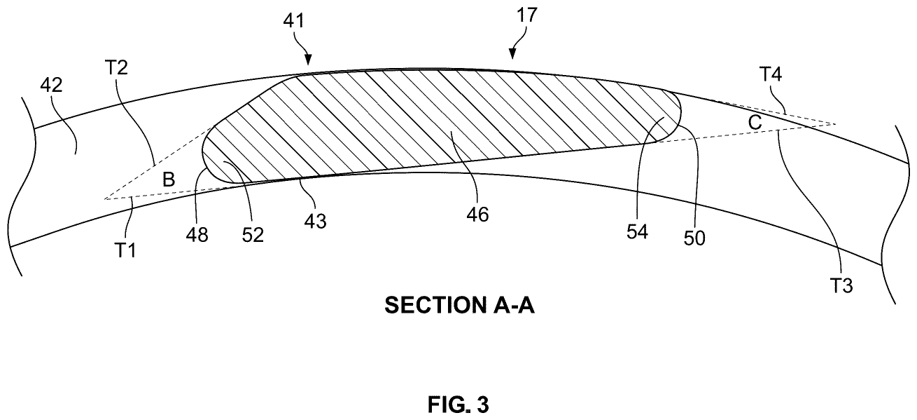

Referring to FIGS. 2 and 3, each of the plurality of struts 46 extends between edges 36 and 38 in an asymmetric or non-linear shape relative to the edges 36 and 38. In one aspect, the struts 46 have a cross-section that may be hydrofoil-like along the entire length of the strut from edge 38 to edge 36. The hydrofoil-like cross-section extends to the end ring 40. Referring to FIG. 2, the cross-section A-A of strut 46 allows the cross-sectional profile of the strut 46 to be understood relative to the edge 38 from which the strut 46 extends. In this aspect, the horizontal cross-section of each strut 46 has a hydrofoil-like profile as illustrated in FIG. 3. Although the applicants do not wish to be held to a particular theory, the hydrofoil-shaped cross-section of the struts 46 of the pump housing 12 may reduce high wall shear stress of the blood flowing through the outlet openings 30 and may also prevent significant blood recirculation as the blood exits through the plurality of outlet openings 30 of the pump housing 12 of the heart pump assembly 10. This, in turn, may reduce hemolysis that may be caused due to placement of the struts in the stream of blood flowing from the plurality of outlet openings 30 of the outlet portion 17 of the pump housing 12 of the heart pump assembly 10. Said another way, the outlet portion 17 of the pump housing 12 with the strut configuration described herein may impart less turbulence to the blood flowing past the struts compared with conventional strut designs that lack a hydrofoil-like profile with filleted leading and trailing edges. One skilled in the art will recognize that reducing the flow turbulence may result in laminar flow of the blood flowing past the struts 146, that will place less stress on red blood cells in the blood and reduce hemolysis.

FIG. 3. illustrates a portion of a cross-sectional view of the strut 46 extending from the edge of body 42 of outlet portion 17 of the pump housing 12. The strut 46 spans between a leading edge 48 and a trailing edge 50. In one aspect, both edges 48, 50 are filleted. Filleted edges reduce flow loss on the blood as it flows past the struts, which flow loss may impede blood flow and induce hemolysis. Filleted edges may also mitigate hemolysis of red blood cells that are carried into contact with the struts as the blood flows past the struts. For reference, the upper perimeter 41 of the edge of the body 42 is referred to as the top side of strut 46 cross-section. The lower perimeter 43 of the edge of the body 42 is referred to as the bottom side of strut 46 cross-section. As can be seen in FIG. 3, the top side of the leading edge 48 of the strut 46 may be shaped so that the leading edge extends away from the upper perimeter 41 of the edge of the body 42. Similarly, the trailing edge 50 may be shaped so that it extends away from the lower perimeter 43 of the edge of the body 42, thereby allowing each strut 46 to have a hydrofoil profile.

As can be seen from the cross-sectional view in FIG. 3, the cross-sectional shape of the strut 46 resembles a hydrofoil profile at least over a leading-edge portion 52 and a trailing edge portion 54 of the strut 46. As illustrated, the strut 46 may be rounded at the leading edge 48, then increases in width rearwardly to span from the outer perimeter 41 to the inner perimeter 43 of the edge of the body 42 before decreasing in width towards the trailing edge 50, which may also be rounded. Both the leading edge 48 and the trailing edge 50 are shaped such that they define or form an acute angle. Specifically, the angle of the leading edge 48 of the strut 46 may be represented by the tangent lines T1, T2 that extend from opposite sides of the leading edge 48. Lines T1 and T2 are extended to form an apex, and the leading-edge angle is the angle B. In one aspect, the strut 46 may be configured such that the leading-edge angle B is from about 30 degrees to about 70 degrees. “About” as used herein is about ten percent of the value to which the term is applied. In one aspect, the leading-edge angle B may be about 20 degrees to about 45 degrees. In a further aspect, the leading-edge angle may be about 30 degrees to about 40 degrees. The tangent lines T3, T4 that extend from opposite sides of the trailing edge 50 are extended to form an apex in order to illustrate a trailing edge angle C, which, in one aspect, may be about 5 degrees to about 45 degrees. The trailing edge angle C may be smaller than the leading-edge angle B. Such an angle for the strut balances the strength of the strut 46 with its benefits of reducing wall shear stress to the blood that flows past the strut. In one aspect, each fillet of the leading edge 48 and the trailing edge 50 has a radii of about 0.05 inches (about 1.25 mm). Although, in the illustrated aspect, the outlet 17 of the pump housing 12 includes three struts 46, it is contemplated that the outlet 17 of pump housing 12 may include any suitable number of struts 46.

The heart pump assembly 10 is made of one or more materials having suitable properties for a desired application, including strength, weight, rigidity, etc. In one aspect, thermoplastics (e.g., polypropylene, polyethylene, etc.) are used to form the blood inlet 16, atraumatic tip 18, and pump housing 12.

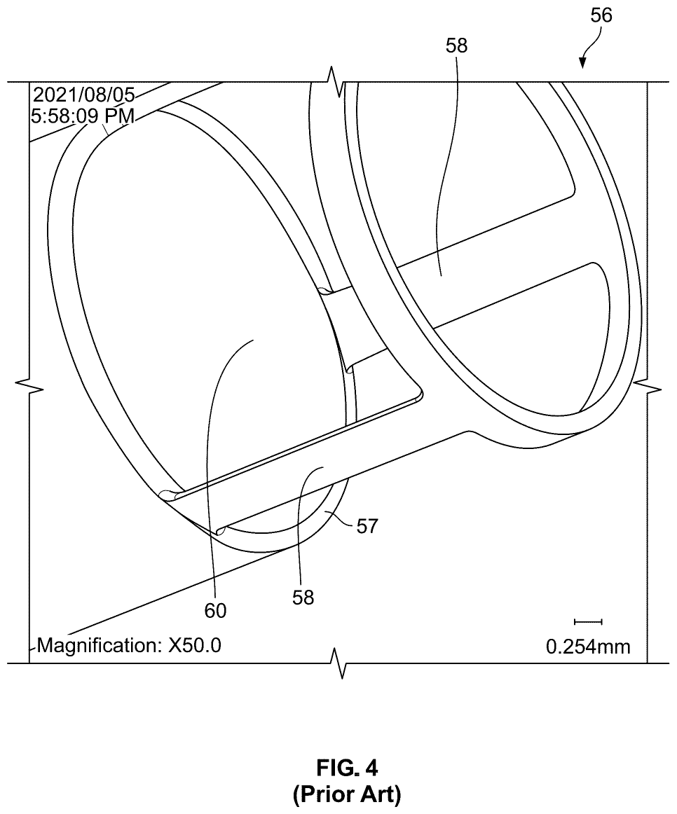

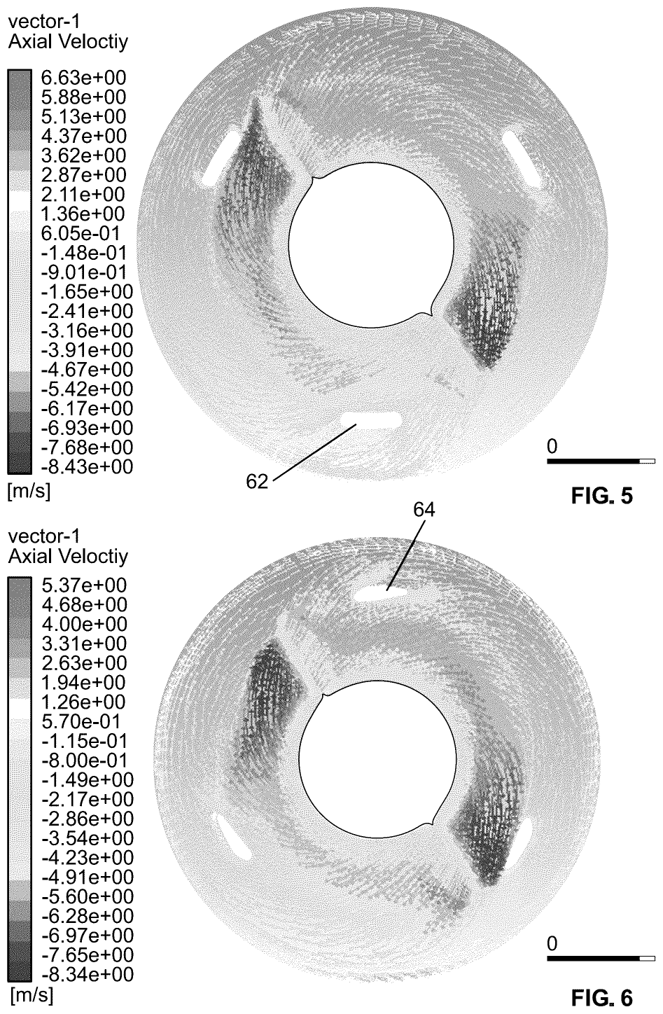

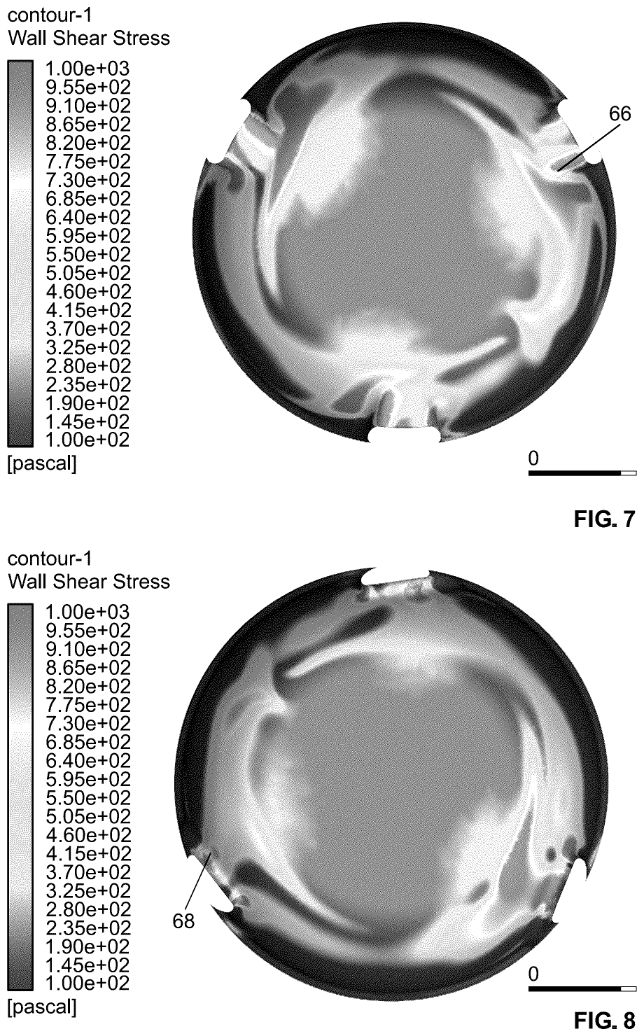

FIG. 4 is a perspective view of an exemplary prior art pump housing 56 of a heart pump assembly. The prior art outlet portion 56 of the pump housing has a plurality of struts 58 that are symmetric in their length and with respect to the body 57 of the outlet 59 from which the struts 58 extend, instead of hydrofoil-like in length as are the struts 46 in the outlet 17 of the pump housing 12 described herein. The design of the prior art pump outlet 56 causes large blood flow recirculation as blood passes through the outlets 60 of the outlet 56 of the pump housing, as shown at the location 62 in FIG. 5, compared to the blood flow recirculation as blood passes through the outlet openings 30 of the improved outlet 17 of the pump housing 12 for the assembly described herein, as shown at the location 64 in FIG. 6. Also, the blood flow at the exit of the outlet 56 of the prior art pump housing has some turbulence at the location 62 in FIG. 5. Further, the design of the outlet 56 of the prior art pump housing produces high wall shear stress as blood passes through the outlets 60 of the outlet 56 of the pump housing, as shown at the location 66 in FIG. 7, compared to the wall shear stress produced by the improved outlet 17 of the pump housing 12 described herein, as shown at the location 68 in FIG. 8.

As stated above, the design and construction of the plurality of struts 46 of the outlet 17 of the pump housing 12 of the heart pump assembly 10 as described herein effectively reduces the wall shear stress of blood and blood flow recirculation that occurs as the blood exits through the plurality of outlet openings 30 of the outlet 17 of the pump housing 12 of the heart pump assembly 10. As stated above, outlet housing with the struts described above may reduce hemolysis in the bloodstream. Specifically, the improved design of the pump housing 12 having a plurality of struts 46 with each strut having a hydrofoil-like profile may reduce turbulence in blood flow at the outlet openings 30 of the pump housing 12. This may reduce hemolysis in the patient's bloodstream. Such laminar flow within the pump housing 12 of the heart pump assembly 10 is illustrated in FIGS. 6 and 8.

Described herein is a percutaneously insertable blood pump assembly that may have a pump section; a catheter attached to a proximal end of the pump section; a first apertured section distal to the pump section and in fluid communication therewith; a cannula affixed to and in fluid communication with the first apertured section; and a second apertured section distal to the cannula and in fluid communication therewith. In one aspect, one of the first and second apertured section is a blood outlet from the cannula. In a further aspect the blood outlet may have a body portion, an apertured portion, and a ring portion wherein a plurality of apertures in the apertured portion may be separated and defined by a plurality of struts extending from and joining the body portion to the ring portion. In a further aspect the struts are asymmetric along their length from the body portion of the outlet to the ring portion of the outlet.

In any of the above aspects, the blood outlet may be the first apertured section outlet or the second apertured section. In any of the above aspect, the asymmetric struts may have a cross-section that may have a leading edge and a trailing edge, the leading edge forming a first angle and the trailing edge forming a second angle. In any of the above aspects, the body portion of the blood outlet may have an outer perimeter and an inner perimeter and the leading edge of the strut is removed from the outer perimeter of the body portion and the trailing edge portion is removed from the inner perimeter of the body portion. In a further aspect the first angle of the leading edge and the second angle of the trailing edge may form acute angles. In a further aspect, the first angle of the leading edge is about 10 degrees to about 70 degrees or about 20 degrees to about 45 degrees or about 30 degrees to about 40 degrees.

In a further aspect, the second angle of the trailing edge is about 5 degrees to about 45 degrees or about 20 degrees.

In a further aspect, the percutaneously insertable blood pump assembly, may have at least one of the leading edge and the trailing edge that is filleted or rounded or a combination of filleted and rounded.

In a further aspect, the percutaneously insertable blood pump assembly may have a filleted edge with a radius of about 1.25 mm. In a further aspect, both the leading edge and the trailing edge of the percutaneously insertable blood pump assembly are filleted or rounded or a combination of filleted and rounded.

In a further aspect, the struts of the percutaneously insertable blood pump may have a hydrofoil-like cross-section along at least a portion of their length from the body portion of the outlet to the ring portion of the outlet or may have a hydrofoil-like cross-section along an entire length from the body portion of the outlet to the ring portion of the outlet. In a further aspect, the plurality of apertures of the percutaneously insertable blood pump may have dimensions and the dimensions of each aperture are about the same. In a further aspect, the percutaneously insertable blood pump assembly may have an atraumatic tip distal to the second apertured section.

As stated previously herein, the outlet as described herein may be at the distal end of the assembly and not attached to the pump housing as described herein, if the pump is configured to work in the right side of the heart instead of the left side of the heart.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

1. A percutaneously insertable blood pump assembly comprising:
a pump section;
a catheter attached to a proximal end of the pump section;
a first apertured section distal to the pump section and in fluid communication therewith;
a cannula affixed to and in fluid communication with the first apertured section; and
a second apertured section distal to the cannula and in fluid communication therewith;
wherein one of the first and second apertured section is a blood outlet from the cannula;
wherein the blood outlet comprises a body portion, an apertured portion, and a ring portion wherein a plurality of apertures in the apertured portion are separated and defined by a plurality of struts extending from and joining the body portion to the ring portion, wherein each of the plurality of struts is asymmetric along its length from the body portion of the outlet to the ring portion of the outlet.

2. The percutaneously insertable blood pump assembly of claim 1, wherein the blood outlet is the first apertured section is the blood outlet.

3. The percutaneously insertable blood pump assembly of claim 1, wherein the blood outlet is the second apertured section.

4. The percutaneously insertable blood pump assembly of claim 1, wherein the asymmetric struts have a cross-section comprising a leading edge and a trailing edge, the leading edge forming a first angle and the trailing edge forming a second angle.

5. The percutaneously insertable blood pump assembly of claim 4, wherein the body portion of the blood outlet comprises an outer perimeter and an inner perimeter and wherein the leading edge of the strut is removed from the outer perimeter of the body portion and at least a portion of the trailing edge is removed from the inner perimeter of the body portion.

6. The percutaneously insertable blood pump assembly of claim 4, wherein the first angle and the second angle form acute angles.

7. The percutaneously insertable blood pump assembly of claim 4, wherein the first angle is about 10 degrees to about 70 degrees.

8. The percutaneously insertable blood pump assembly of claim 4, wherein the first angle is about 20 degrees to about 45 degrees.

9. The percutaneously insertable blood pump assembly of claim 4, wherein the first angle is about 30 degrees to about 40 degrees.

10. The percutaneously insertable blood pump assembly of claim 4, wherein the second angle is about 5 degrees to about 45 degrees.

11. The percutaneously insertable blood pump assembly of claim 4, wherein the second angle is about 20 degrees.

12. The percutaneously insertable blood pump assembly of claim 5, wherein the at least one of the leading edge and the trailing edge are filleted or rounded or a combination of filleted and rounded.

13. The percutaneously insertable blood pump assembly of claim 12, wherein the fillet has a radius of about 1.25 mm.

14. The percutaneously insertable blood pump assembly of claim 5, wherein both the leading edge and the trailing edge are filleted or rounded or a combination of filleted and rounded.

15. The percutaneously insertable blood pump assembly of claim 1, wherein each strut of the plurality of struts has a hydrofoil-like cross-section along at least a portion of their length from the body portion of the outlet to the ring portion of the outlet.

16. The percutaneously insertable blood pump assembly of claim 1, wherein each strut of the plurality of struts have a hydrofoil-like cross-section along an entire length from the body portion of the outlet to the ring portion of the outlet.

17. The percutaneously insertable blood pump assembly of claim 1, wherein the plurality of apertures have dimensions and the dimensions of each aperture are about the same.

18. The percutaneously insertable blood pump assembly of claim 1, further comprising an atraumatic tip distal to the second apertured section.
